(19) United States
(12) Patent Application Publication
(10) Pub. No.: US 2007/0118129 A1
(43) Pub. Date: May 24, 2007

(54) Implant fixation methods and apparatus

(75) Inventors: Robert Fraser, Myrtle Bank (AU); Rehan Khanzada, Middleboro, MA (US); Michael J. O'Neil, West Barnstable, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: DePuy Spine, Inc., Raynham, MA (US)

(21) Appl. No.: 11/285,503

(22) Filed: Nov. 22, 2005

(51) Int. Cl. A61B 17/80; A61B 17/84

(52) U.S. Cl. 606/71; 606/72; 606/70

(57) ABSTRACT

Various exemplary methods and devices are provided for fixing an implant to native tissue, such as, bone. The devices can include first and second plate bodies, each having an upper surface and a lower tissue contacting surface. The bodies can provides a large contact area for fixing the tissue implant to bone, such that the implant can be securely held in position without requiring penetration of the implant. In one embodiment, the plate bodies can include at least one aperture for receiving a fixation device and a tissue implant receiving opening through which a tissue implant can be threaded prior to fixing the plate bodies to bone.

### BACKGROUND OF THE INVENTION

Disease, advancing age, and trauma can lead to changes in various bones, discs, joints, and ligaments of the body. Some changes and trauma often manifest themselves in the form of damage or degeneration to a spinal disc. This condition often results in chronic back pain, which can be anywhere from mild to severe. This pain can sometimes be eliminated by removing the disc tissue between adjacent vertebral bodies and replacing it with a prosthetic device.

One type of procedure is spinal fusion, in which two adjacent vertebral bodies are jointed together after removing the intervening intervertebral disc. A prosthetic device is usually placed between the two adjacent vertebral bodies, in place of the removed disc, to fill the space left by the removed disc and to allow bone to grow between the two vertebral bodies. Alternatively, proposals have been made to replace the defective disc with an artificial disc that preserves the natural mobility between adjacent vertebral bodies. For example, such prostheses can include first and second plates for fixing to adjacent vertebral bodies, the plates having low friction contact surfaces that allow articulation.

As part of the surgical procedure to replace a disc, the soft tissue connecting adjacent vertebral bodies is at least partially cut-away. This can cause a loss of stability, particular where a mobility retaining prosthesis is utilized. To replace the function of the connective tissue, a tissue implant can be implanted. For example, a tissue implant can fixed at a first end to a first vertebral body and fixed at a second end to second vertebral body. To fix the tissue implants to bone, a surgeon can drive a screw, tack, or staple through the tissue implant and into the native tissue.

One drawback of such procedures is that the tissue implant can be weakened by the fixation procedure. When the screw, tack, or staple is driven through the implant it creates a weak spot, which may tear under load. For example, the screw or staple could be pulled through the tissue implant when tension is applied. Another drawback of conventional fixation techniques is that the fixation devices hold only a minimal portion of the implant in contact with the native tissue surface.

Accordingly, there remains a need for improved devices for fixing tissue implants, particularly, methods and devices that can fix tissue while causing a minimum weakening of the implant and/or that can provide improved contact between the tissue implant and a native tissue surface.

### SUMMARY OF THE INVENTION

Described herein are methods and apparatus for fixing implants to bone. Unlike traditional fixation devices, such as bone screws or staples, the device described herein can include a large surface area for holding an implant in place. In addition, in at least one embodiment, the device is adapted to hold an implant without penetrating the implant. For example, an implant can be fixed in place between the device and a native tissue surface by implanting a bone screw through a portion of the device that is spaced from the tissue implant.

In one embodiment, the tissue fixation device includes a first plate body having an upper surface and a lower implant contacting surface and a second plate body having an upper implant contacting surface and a lower native tissue contacting surface. The first and second plate bodies can also include at least one aperture for receiving a fixation element. In use, an implant can be positioned between the first and second plate bodies and/or between the second plate body and a native tissue surface. A fixation element can be implanted through the at least one aperture to fix the implant in place.

In one aspect, the aperture(s) is adapted to receive a fixation element selected from the group consisting of a pin, screw, tack, staple, tine, anchor, expansion bolt, rivet, and combinations thereof. In one exemplary embodiment, each aperture is shape to receive the head of a fixation element.

The first and/or second plate body can also include an implant receiving opening defined by an elongate slot. In use, a tissue implant can be positioned at least partially in the elongate slot and fixed between the plate bodies and/or a tissue surface. For example, a tissue implant can extend through an elongate slot in the second plate body and be fixed between the first and second plates and between the second plate and a native tissue surface.

The plate bodies described herein can be shaped for positioning on a tissue surface such as, for example, a vertebral body. For example, the device can have a curvature that corresponds to the curvature of the surface of a vertebral body. In addition, or alternatively, the plate bodies can be flexible or malleable. When the device is implanted, the plate body can deform and/or bend to match the curvature of a tissue surface. In another aspect, the plate bodies are shaped and sized for positioning on a single vertebral body. For example, the apertures can be spaced for implantation in a single vertebral body.

The plate bodies can further include features to assist with fixing a tissue implant. In one aspect, the lower tissue contacting surface of the second plate body includes a recess for seating an implant. In another aspect, the device can include surface features for gripping an implant.

In another embodiment described herein a method of implanting a soft tissue implant is provided. The method can include the step of providing a first plate body having an upper surface and a lower implant contacting surface and a second plate body having an upper implant contacting surface and a lower tissue contacting surface. An implant can be positioned between the first and second plate bodies and/or between the second plate body and a native tissue surface. Fixing the first and second plates to a hard tissue surface can fix the implant between the first and second plate bodies and between the second plate body and a native tissue surface.

In one aspect, the method further includes the step of threading the implant through an implant receiving slot of a second plate body. In another aspect, the method includes the step of implanting the first and second plate bodies on a single vertebral body.

Further features of the invention, its nature and various advantages, will be more apparent from the accompanying drawings and the following detailed description of the drawings and the preferred embodiments.

### BRIEF DESCRIPTION OF THE DRAWINGS

The invention can be more fully understood from the following detailed description taken in conjunction with the accompanying drawings, in which:

FIG. 1A is a top view of one exemplary embodiment of a tissue fixation device described herein;

FIG. 1B is a side view of the device of FIG. 1A;

FIG. 2 is a cross-sectional view of the device of FIG. 1A implanted on a vertebral body;

FIG. 3A is a top view of another embodiment of a device described herein;

FIG. 3B is a side view of the device of FIG. 3A;

FIG. 3C is a perspective view of the device of FIG. 3A;

FIG. 4A is a top view of yet another embodiment of a device described herein;

FIG. 4B is a perspective view of the device of FIG. 4A;

FIG. 5A is a top view of still another embodiment of a device described herein;

FIG. 5B is a perspective view of the device of FIG. 5A;

FIG. 6A is a top view of another embodiment of a device described herein;

FIG. 6B is a side view of the device of FIG. 6A;

FIG. 6C is a partial cross-sectional view of the device of FIG. 6A;

FIG. 7A is a side view of a two-plate embodiment of a device described herein;

FIG. 7B is a perspective view of the device of FIG. 7A;

FIG. 7C is another side view of the device of FIG. 7A;

FIG. 8A is a perspective view of an embodiment of a device described herein that is adapted for positioning on an edge of a vertebral body;

FIG. 8B is a side view of the device of FIG. 8A;

FIG. 8C is another side view of the device of FIG. 8A;

FIG. 9 is a perspective view of the device of FIG. 8A with an implant positioned therein;

FIG. 10A is a perspective view of one embodiment of a single fixation device described herein;

FIG. 10B is a side view of the device of FIG. 10A;

FIG. 10C is another side view of the device of FIG. 10A;

FIG. 11A is a perspective view of another embodiment of a device described herein;

FIG. 11B is a side view of the device of FIG. 11A;

FIG. 11C is another side view of the device of FIG. 11A;

FIG. 12A is a perspective view of yet another embodiment of a device described herein;

FIG. 12B is a top view of the device of FIG. 12A;

FIG. 13A is a side view of one embodiment of a device described herein that includes mating features;

FIG. 13B is a perspective view of the device of FIG. 13A;

FIG. 14A is a top view of one embodiment of a device described herein that includes an extension portion;

FIG. 14B is a perspective view of the device of FIG. 14A;

FIG. 14C is a side view of the device of FIG. 14A;

FIG. 15 is a side view of the device of FIG. 14A implanted on a vertebral body;

FIG. 16A is a perspective view of one embodiment of a device described herein that includes a hinge;

FIG. 16B is a top view of the device of FIG. 16A;

FIG. 16C is a cross-sectional view of the device of FIG. 16A;

FIG. 17A is a cross-sectional view of another embodiment of a device described herein;

FIG. 17B is another cross-sectional view of the device of FIG. 17A

FIG. 18 is a top view of an embodiment of a device described herein that includes multiple implant receiving openings;

FIG. 19A is a cross-sectional side view of another embodiment of a device described herein; and

FIG. 19B is a perspective view of the device of FIG. 19A.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention

Various exemplary methods and devices are provided for fixing an implant to native tissue, such as, bone. One such device includes at least one plate having a body with an upper surface and a lower tissue contacting surface. The body provides a large contact area for fixing the tissue implant to bone, such that the implant can be securely held in position without requiring penetration of the implant. In one embodiment, the plate body can include at least one aperture for receiving a fixation device and a tissue implant receiving opening through which a tissue implant can be threaded prior to fixing the plate to bone. A tissue implant can be positioned in the implant receiving opening and then fixed to bone by implanting a bone screw in the at least one aperture. In an alternative embodiment, two plate bodies can be used to fix a tissue implant in place. For example, first and second plate bodies can be implanted on native tissue with a tissue implant fixed therebetween. In yet another embodiment, the plate body can include two segments joined with a hinge. At least one of the segments can be pivoted to close a portion of an implant therebetween and to fix the implant in position.

While the methods and devices are described herein with respect to implantation on a vertebral body, one skilled in the art will appreciate that the devices can be implanted on a variety of native tissue surfaces for fixing a variety of tissue implants. Exemplary tissue surfaces include the variety of hard tissue surfaces that may or may not be covered with soft tissue. In particular, the terms “hard tissue surface,” “bone,” and “vertebral body” do not exclude structures having a native soft tissue coating or layer that may, for example, include cartilage, tendons, ligaments, meniscus, other soft tissue structures.

Unlike conventional devices, such as bone screws, the apparatus described herein can fix a tissue implant to bone without penetrating the implant. Conventional tissue implant fixation procedures can weaken a tissue implant, specifically in the region where the implant is penetrated. In addition, the plates described herein provide a large surface area, which can join the tissue implant to bone and provide additional securement. Thus, even if an implant is penetrated by a securement device described herein, the implant contacting surfaces of the device can help to prevent tearing of the tissue implant when tension is applied to the implant. The devices described herein can also allow an implant to be tensioned prior to fixation.

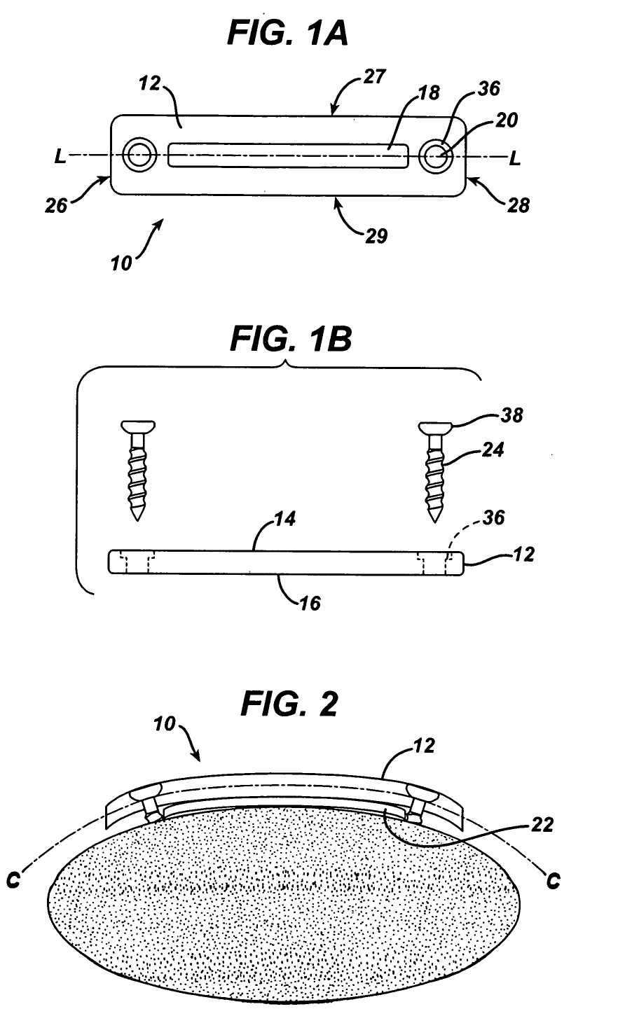

In one embodiment, illustrated in FIGS. 1A through 6C, a single plate device 10 is described. As shown in FIGS. 1A through 1B, plate 10 can include body 12 having a top surface 14 and a lower tissue contact surface 16. Body 12 can also include an implant receiving opening 18 and apertures 20. In use, an implant 22 can extend across bottom surface 16 of the body, through implant receiving opening 18, and can wrap around top surface 14. The implant is fixed in place by pinning the tissue implant between lower surface 16 and a tissue surface as shown in FIG. 2. For example, with implant 22 in position, bone screws 24 can be implanted through apertures 20 to fix body 12 and implant 22 in place.

Body 12 can have a variety of shapes and sizes, including, for example, an elongate shape extending along a longitudinal axis L from a first end 26 to a second end 28 and can include lateral sides 27, 29. One skilled in the art will appreciate that body 12 could alternatively have a variety of other shapes such as a circular, triangular, or irregular shape. In one aspect, the shape and size of body 12 is adapted for positioning on a vertebral body, such as on a single vertebral body. To assist with fixation, body 12 can include a curvature adapted to match the anatomical curvature of the surface onto which device 10 will be implanted (i.e., the curvature of the surface of a vertebral body). FIG. 2 illustrates body 12 having a curvature C along longitudinal axis L that allows body 12 to conform to the curvature of a boney tissue (e.g., vertebral body). Body 12 can also, or alternatively, have a curvature along an axis that is different from longitudinal axis L, such as, for example along a transverse axis. One skilled in the art will appreciate that the shape and size of body 12 can be adapted to various surfaces and curvatures of vertebral bodies as discussed in more detail below.

Body 12 can be, in one embodiment, flexible or semi-rigid and/or deformable, such that device 10 will conform to the tissue surface on which it is implanted, thereby enhancing contact and securement between native tissue and the tissue implant. Alternatively, body 12 can be rigid and/or non-deformable. One skilled in the art will appreciate that body 12 can be produced from the variety of materials used in orthopaedic or implantable devices, such as, for example metals, polymers, and/or natural materials. Examples include Ti64, CoCr, resorbable and non-resorbable polymers, allografts, autografts, xenografts, and combinations thereof.

Tissue receiving opening 18 in body 12 can be adapted for receiving a variety of tissue implants. The shape and size of opening 18 can be adapted to the implant shape and size, including generally planar implants such an implant 22. In one aspect, opening 18 is defined by a longitudinally extending slot that allows passage of at least a portion of implant 22 therethrough. One skilled in the art will appreciate that opening 18 can have a variety of alternative shapes and sizes that could receive implants of various shapes and sizes (e.g., implants having a circular, triangular, or irregular shape). In addition, slot shaped opening 18 could receiving a variety of differently sized and shaped implants.

To assist with threading an implant through opening 18, the top and or bottom surfaces of body 12 can include a gradient around opening 18 that slopes toward the opening. In use, the sloped surfaces can help direct the implants into opening 18. In addition, once the implant is positioned within opening 18, the sloped surfaces can help to reduce abrading and/or point loading of the implant. Other surface of body 12 can also include a gradient to reduce point loadings, such as, for example, at least one of lateral sides 27, 29.

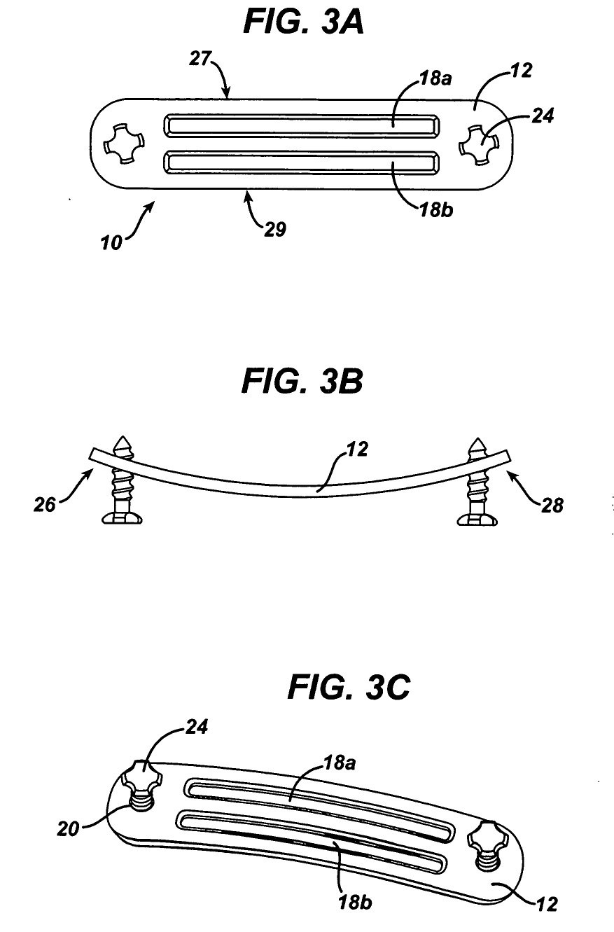

As shown in FIG. 1A, a single opening 18 can extend along the longitudinal axis L. Alternatively, opening 18 could be offset from longitudinal axis L and/or be positioned at an angle with respect to axis L. In another embodiment, illustrated in FIGS. 3A through 3C, body 12 could include more than one opening. Device 10 can include one, two, three, or more than three openings 18, each adapted to receive at least a portion of an implant. The two-opening embodiment shown in FIGS. 3A through 3C includes two parallel openings 18a, 18b through which an implant could be threaded. For example, an implant could extend from bottom surface 16 through opening 18a, over top surface 14, and back through opening 18b. In the case of three or more openings, the implant could be threaded through the three or more successive openings. While openings 18a, 18b are illustrated in a parallel configuration, one skilled in the art will appreciate that openings 18a, 18b could alternatively extend at an angle with respect to one another.

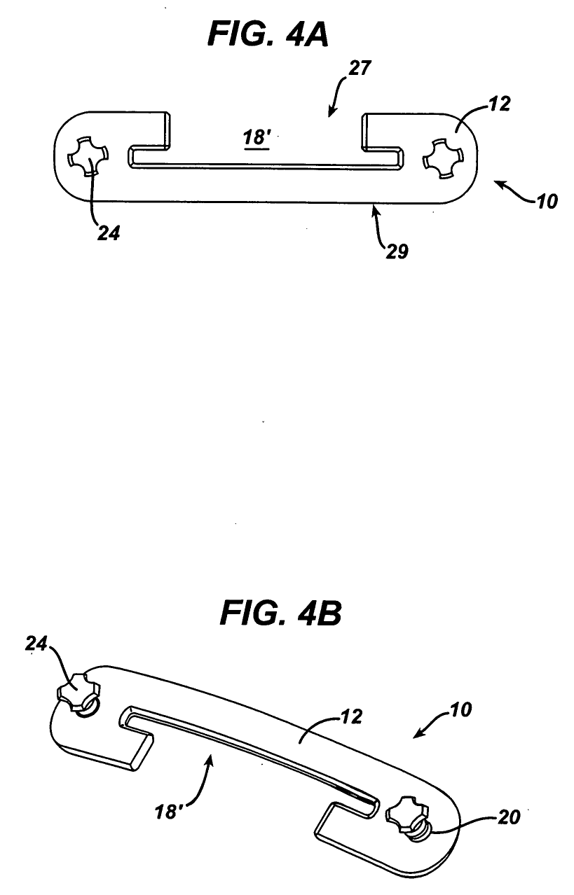

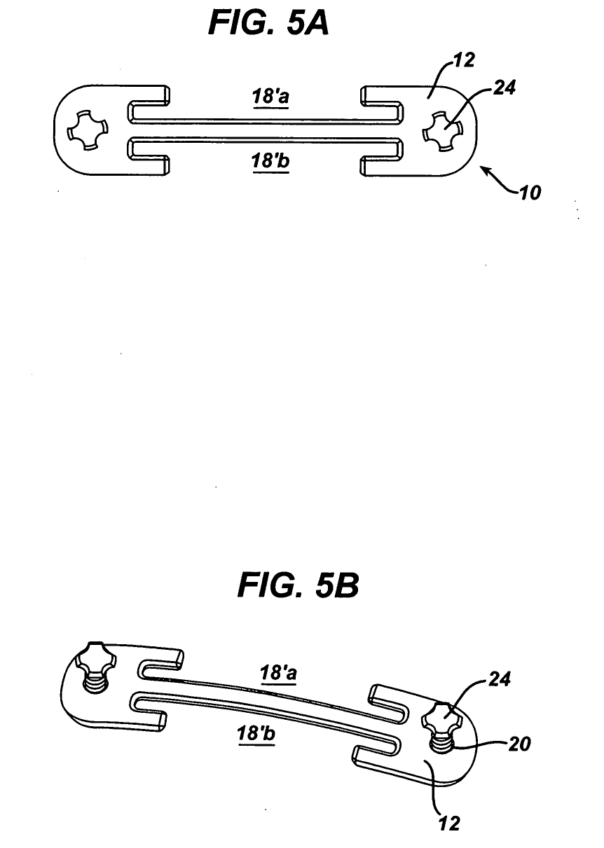

FIGS. 4A and 4B illustrates an opening 18′ that extends longitudinally as well as transversely. For example, opening 18′ extends to the lateral edge 27 of body 12 to provide an “open” configuration. In an alternative embodiment, illustrated in FIGS. 5A and 5B, two openings 18′a, 18′b are present and each extends laterally to lateral sides 27, 29 and include non-contiguous lateral edges 27, 29. The implant can be seated in openings 18′a, 18′b without having to thread the end of the implant through the openings 18′a, 18′b.

As shown in FIGS. 1A through 6C, opening 18, 18′ and apertures 20 are positioned such that when a fixation device is inserted through the apertures, the fixation device does not penetrate an implant. In one aspect, apertures 20 are positioned longitudinally with respect to opening 18 as shown in FIGS. 1A through 5C. When the implant is threaded through opening 18 the implant only covers a portion of lower surface 16, leaving unobstructed the lower surface adjacent to body ends 26, 28. Fixation device can then be implanted through apertures 20 without impinging on the implant.

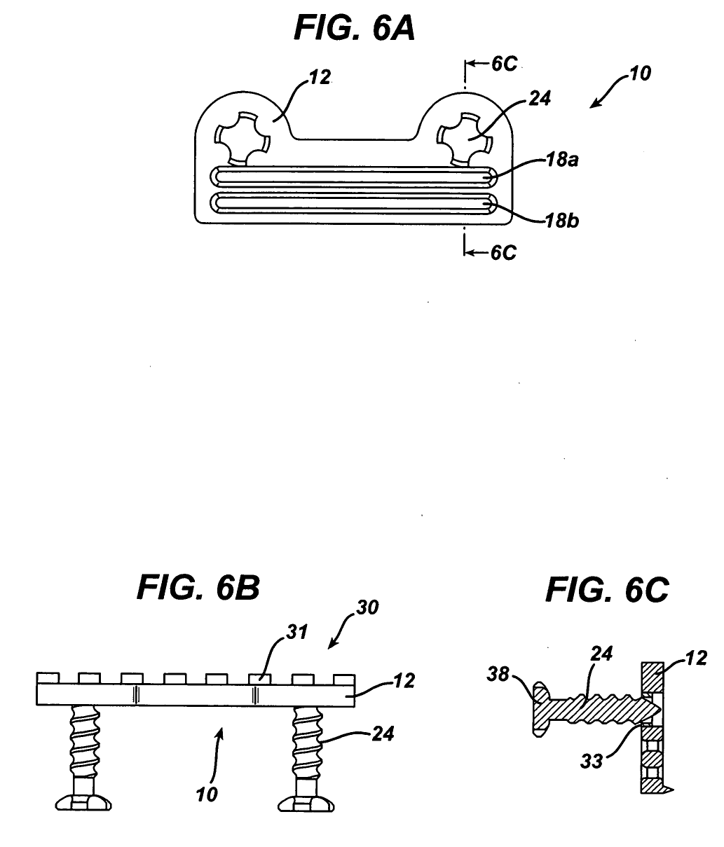

Alternatively, as shown in FIGS. 6A through 6C, apertures 20 can be positioned transversely with respect to openings 18a, 18b. An implant can be threaded through and wrapped around a portion of body 12, leaving a portion of upper and lower surfaces 14, 16 adjacent to lateral edge 27 unobstructed. In use, fixation devices can be inserted through apertures 20 without penetrating an implant seated within openings 18a, 18b.

Body 12 of device 10 can include any number of apertures 20. In one embodiment, body 12 includes a single aperture (not shown). Alternatively, body 12 can include two or more apertures 20. One skilled in the art will appreciate that the quantity of apertures can depend on a number of factors including, the degree of securement required, the securement surface, the expected strains and stresses, the type of fixation devices used, and placement of the device.

Apertures 20, in one embodiment, are each adapted to receive a different fixation device. In use, the number of apertures can correspond to the number of fixation devices used to implant the device. For example, where two apertures are used, two bone screws are used to implant device 10. In one aspect, top surface 14 of body 12 adjacent to apertures 20 can have a shape suitable to receive the fixation device, such as, for example a bone screw. FIGS. 1A and 1B illustrate body 12 with recessed area 36 around apertures 20 that has a shape corresponding to a head 38 of bone screws 24. When bone screws 24 are positioned in apertures 20, bone screw head 38 is seated at least partially within recessed area 36.

Alternatively, or additionally, device 10 can include features to mate a bone screw with body 12. For example, bone screws 24 can be held within apertures 20 by way of a snap-ring, webs, locking screw, snap-fit, friction fit, or other alternative configuration. FIG. 6C illustrates a cross-section of a portion of plate body 12. Seated within plate body 12 is a retaining ring 33 that is adapted to mate with bone screw 24. As bone screw 24 is implanted through aperture 20, the head 38 of bone screw 24 will mate with retaining ring 33 and be held in position. One skilled in the art will appreciate that device 10 can include a variety of features for mating body 12 with fixation devices. Alternatively, bone screws 24 can be pre-assembled with device 10 and permanently positioned within body 12.

A variety of fixation devices can be used to implant device 10. In one embodiment, the fixation devices are bone fixation elements, including for, example, bone screws, pins, tines, wires, rivets, anchors, expansion bolts, and combinations thereof. One skilled in the art will appreciate that a wide variety of fixation devices can be used with the methods and apparatus described herein.

Body 12 can include a variety of features to assist with implantation/securement and/or tissue in-growth. In one aspect, body 12 includes surface features 30 that will contact native tissue when device 10 is implanted. Such surface features can include, for example, barbs, tines, fins, ribs, securement ridges, porous beading, and/or other coatings/treatments that promote soft or boney tissue apposition, integration, and/or plate resporbtion. Surface features 30 can be positioned, for example, on the tissue contacting tissue surface of device 10, i.e., on lower surface 14. In addition, such surface features can be positioned on body 12 such that they contact implant 22. For example, such surface features can be positioned on top surface 14 and/or bottom surface 16 where the implant will contact device 10. In one aspect, were surface features 30 contact implant 22, the surface features are adapted such that they do not penetrate the implant.

FIG. 6B illustrates body 12 with surface features 30 (i.e., ridges 31) positioned on bottom surface 16 of device 10. Ridges 31 are positioned transversely across body 12 and extend the full length of bottom surface 16. In use, ridges 31 can contact native tissue, as well as, an implant positioned between bottom surface 16 and native tissue.

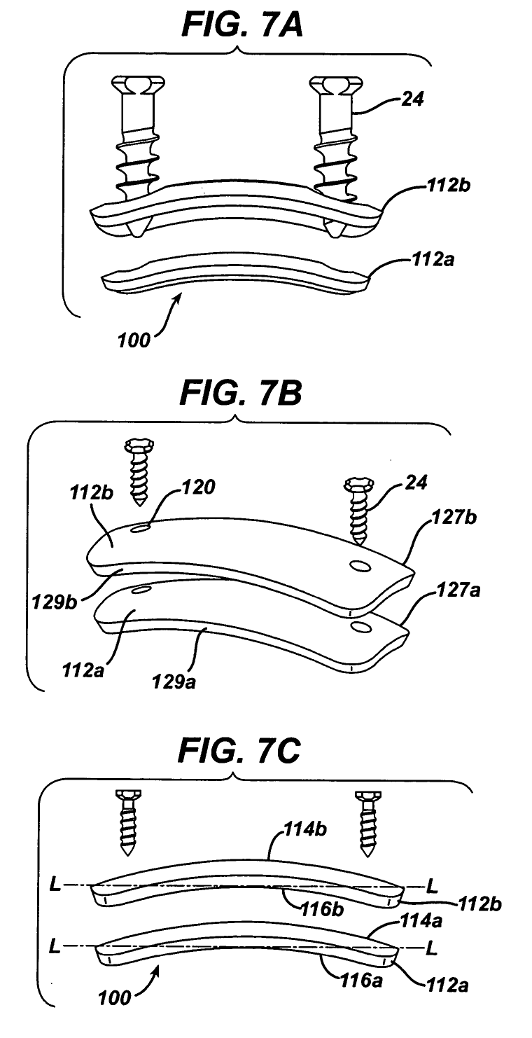

In another embodiment described herein, device 100 includes two or more plates that can work together to fix a tissue implant. Device 100, as shown in FIGS. 7A through 12B, can include a first, bottom plate body 112a and a second, top plate body 112b adapted to receive at least a portion of a tissue implant therebetween. Plate bodies 112a, 112b can provide a large surface area for holding a tissue implant such that the implant can support a maximum load without tearing.

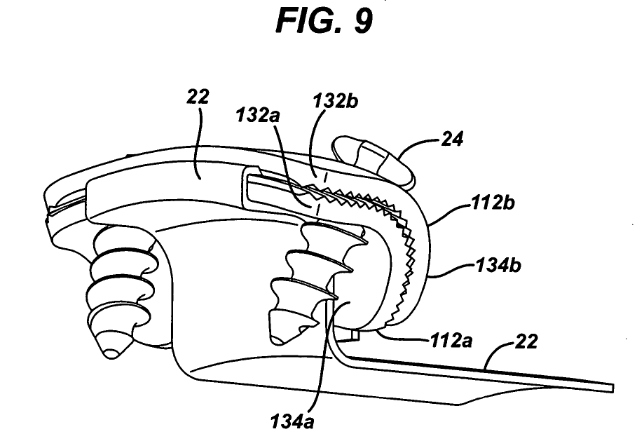

In use, an implant can be fixed between the bottom plate body and native tissue, as well as, between the plate bodies, thereby providing securement of the implant. In one embodiment, the implant extends across a bottom surface 116a of the bottom plate 112a, then wraps around a lateral edge 127a (or 129a) of the bottom plate, and extends between a top surface 114a of the bottom plate and a bottom surface 116b of the top plate 112b as generally shown in FIG. 9. The top and bottom plate are then implanted using fixation devices. In an alternative embodiment, the implant can also, or alternatively, extend through a tissue receiving opening in one of the plates.

7A through 7C illustrate one embodiment of device 100 having plate bodies 112a, 112b of similar size and shape, both including apertures 120 for receiving fixation devices 24. Inserting the fixation devices through apertures 120 implants plate bodies 112a, 112b on native tissue while fixing a tissue implant in position. Bottom plate 112a includes a tissue contacting lower surface 116a and an implant contacting upper surface 114a, while the upper plate 112b includes an implant contacting lower surface 116b and an upper surface 114b. In one embodiment, at least a portion of upper surface 114a can nest within lower surface 116b when the plate bodies are implanted.

aspect, both plate bodies 112a, 112b are similar to body 12 described above and can be shaped and sized according to the anatomical features of the tissue to which they will be implanted. In one embodiment, illustrated in FIGS. 7A through 7C, plate bodies 112a, 112b have an elongate shape with a curvature adapted for positioning on a vertebral body. For example, the plates can have a concave lower surface 116a, 116b and a convex upper surface 114a, 114b, such that a curvature extends along the longitudinal axis L and along an axis perpendicular to the longitudinal axis. In use, the plate bodies can be positioned on a generally convex surface.

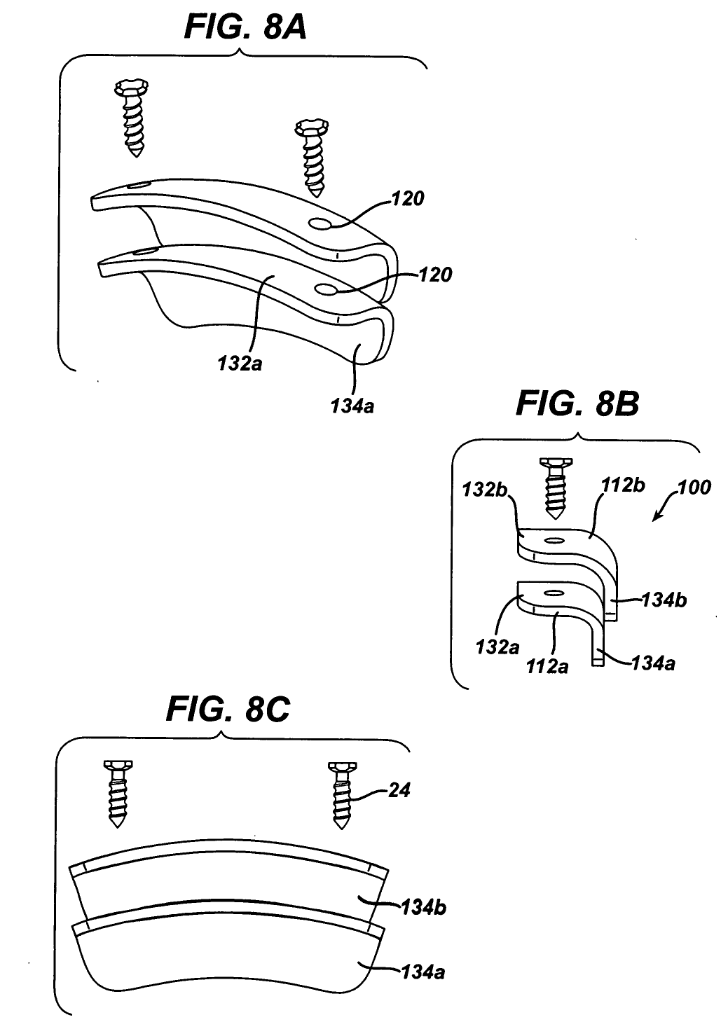

In an alterative embodiment, device 100 can include plate bodies 112a, 112b sized and shaped for implantation on an edge of a vertebral body. Plate bodies 112a, 112b can include a minor curvature along longitudinal axis L and a major curvature along an axis perpendicular to the longitudinal axis, the major curvature corresponding to the curvature of an edge of a vertebral body. FIGS. 8A through 9 illustrates one such embodiment of device 100. Elongate plate bodies 112a, 112b have a major curvature such that the plate bodies include first segments 132a, 132b and second segments 134a, 134b, where the first and second segments are positioned at an angle with respect to one another. In use, the first plate segment 132a, 132b can be positioned on a first surface of a vertebral body and the second plate segment 134a, 134b can be positioned on a second surface of a vertebral body.

FIG. 9 illustrates the plates of FIGS. 8A and 8B implanted on a vertebral body. The curvature of the plate bodies 112a, 112b is generally complementary to the curvature of the vertebral body. One skilled in the art will appreciate that the angle at which segments 132a, 132b are positioned with respect to segments 134a, 134b can be varied according to the shape of the vertebral body. In one aspect, the first and second segments are positioned at an angle with respect to one another that is in the range of about 15 degrees and 150 degrees, and more preferably in the range of about 80 degrees and 130 degrees.

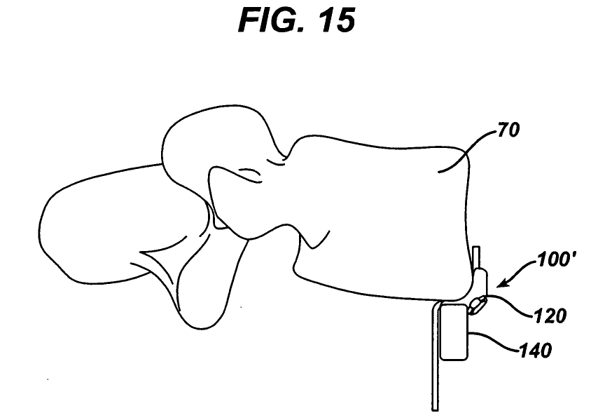

The apertures 120 can be positioned in plates 112a, 112b in a variety of location as described above. For example, FIG. 9 illustrates apertures in first plate segments 132a, 132b. Alternatively, apertures 120 could be positioned closer to the major curvature of plates 112a, 112b and/or in second plate segments 134a, 134b. In one aspect, apertures 120 are positioned in plates 112a, 112b such that fixation elements inserted through apertures 120 can be implanted in the thick cortical rim of a vertebral body. For example, FIG. 15 (discussed below) includes apertures 120 positioned such that bone screws are implanted into the cortical rim and angled away from the disc space. This configuration can provide additional bone depth for fixation element implantation, thereby improving securement of plates 112a, 112b. In addition, locating apertures 120 close to the major curvature can improve surgical access, minimizing the amount of soft tissue damage.

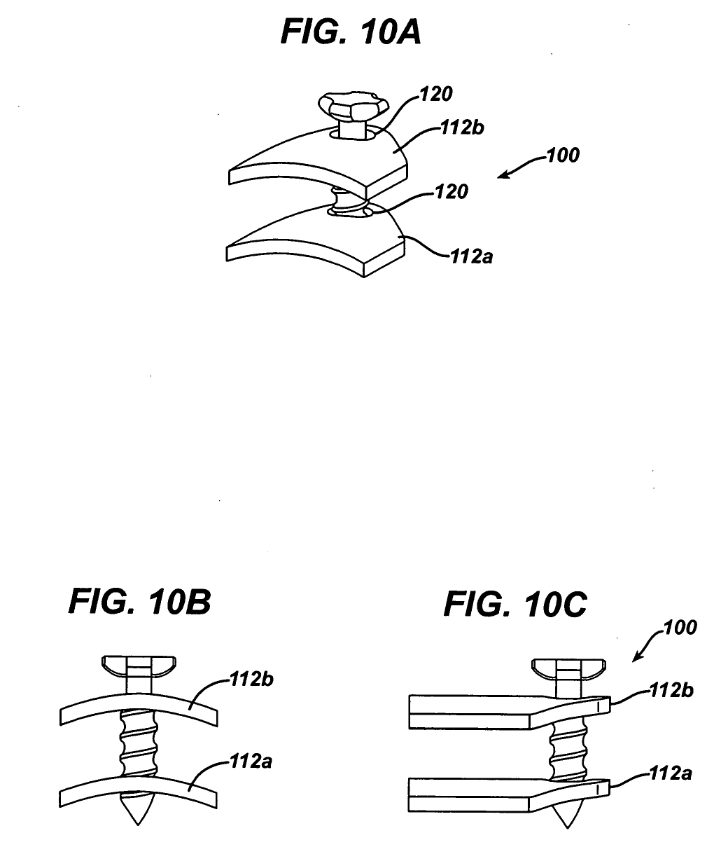

Plates 112a, 112b of device 100 can have a variety of shapes and sizes as mentioned above. FIGS. 10A and 10B illustrate yet another embodiment of plates 112a, 112b having a generally trapezoidal shape and a single aperture 20. In use, an implant can be positioned between plate bodies 112a, 112b and fixed in place with a single fixation device. The small profile of the plate bodies allows for fixation of implants on smaller surfaces and/or for applications where it is preferable to use only a single fixation device.

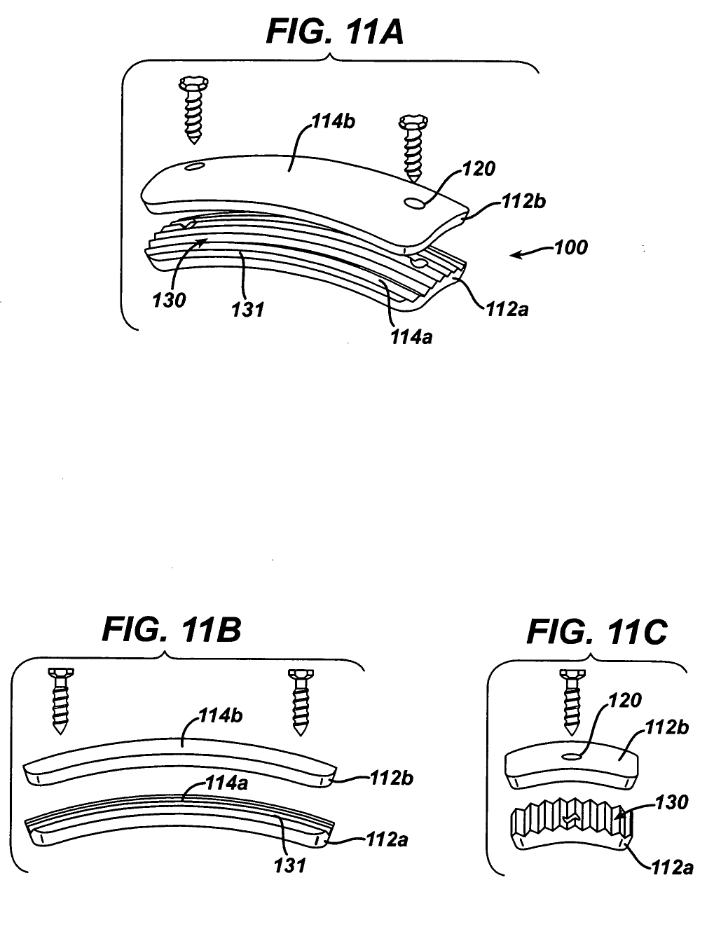

To assist with fixation, plate bodies 112a, 112b can include surface features 130 adapted to grip an implant. FIGS. 11A through 11C illustrate plate bodies 112a, 112b with surface features 130 positioned on the top surface 114a of plate body 112a. As shown, ridges can extend longitudinally across the top of plate 112a and be positioned across the entire surface of the plate. In another example, shown in FIG. 11C, the surface features 130 can extend transversely to the longitudinal axis of the plate body. One skilled in the art will appreciate that ridges 131 can alternatively be positioned on only a portion of the plate surface (not shown). For example, ridges 131 could be positioned only on the portions of the plate surface that will contact an implant positioned between the plates. In addition to top surface 114a, the bottom surface 116b of plate body 12b can include surface features. In one embodiment, top plate 112b can include surface features that are complementary to the surface features on top surface 114a.

Exemplary surface features 130 can include barbs, tines, fins, ribs, securement ridges, porous beading, and/or other coatings/treatments. In one embodiment, surface features 130 are adapted to grip the implant without penetrating the implant. One skilled in the art will appreciate that the “penetrating” nature of surface features can depend on the geometry of the surface features as well as the amount of force which the plates apply to the implant. Conversely, since the implant is pinned at two locations (i.e., between the lower plate and native tissue and between the plate bodies 112a, 112b), implant penetrating surface features can be located on the surface(s) between plate bodies 112a, 112b.

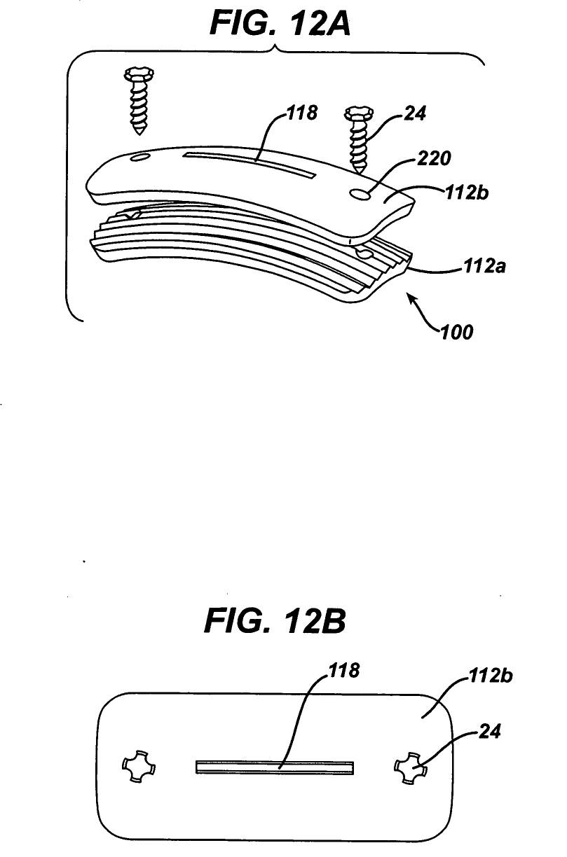

Plate bodies 112a, 112b can include a variety of other features as disclosed with respect to the single plate embodiment described above. In one embodiment, device 100 can include an implant receiving opening 118. FIGS. 12A and 12B illustrate device 100 with opening 118 in upper plate body 112b. In use, an implant can extend between the plate bodies and through opening 118. One skilled in the art will appreciate that plate bodies 112a, 112b can include more than one opening 118.

In another embodiment of device 100, plates 112a, 112b can be adapted to mate with one another. For example, mating features can be positioned plate bodies 112a, 112b to allow mating of the plates to one another after positioning an implant therebetween. The mating features can facilitate implantation of device 100 by keeping apertures 120 of plate bodies 112a, 112b aligned during fixation of the device. Plate bodies 112a, 112b can be mated in a variety of permanent or non-permanent ways, including, for example, snap-fit, friction fit, tongue and groove, crimping (including cold compression welding and/or bending of the plate bodies), welding (including thermal, mechanical, ultrasonic, and RF), and combinations thereof.

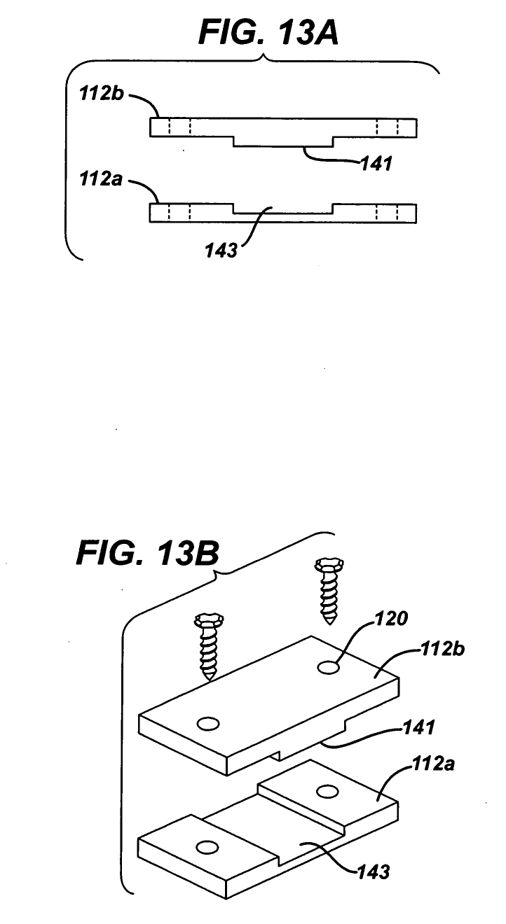

FIGS. 13A and 13B illustrate exemplary plate bodies 112a, 112b having a snap-fit arrangement. Plate body 112b includes a protrusion portion 141 that is adapt to sit within a recess 143. After an implant is positioned in recess 143, protrusion portion 141 of plate 112b can be seated at least partially within recess 143. Protrusion portion 141 can be sized and shaped to snap into recess 143 and thus be held in place. One skilled in the art will appreciate that a variety of other mating techniques can be used with the devices described herein.

In addition or as an alternative to holding plates together, the mating features can assist with alignment. For example, the protrusion/recess of plates 112a, 112b in FIG. 13A and 13B can be sized and shaped such that they will only match up when plates 112a, 112b are properly aligned. The mating features can thereby provide a signal that plates 112a, 112b and apertures 120 are properly aligned. In one aspect, the mating features can be designed to provide tactile and/or auditory feedback when the plates are aligned.

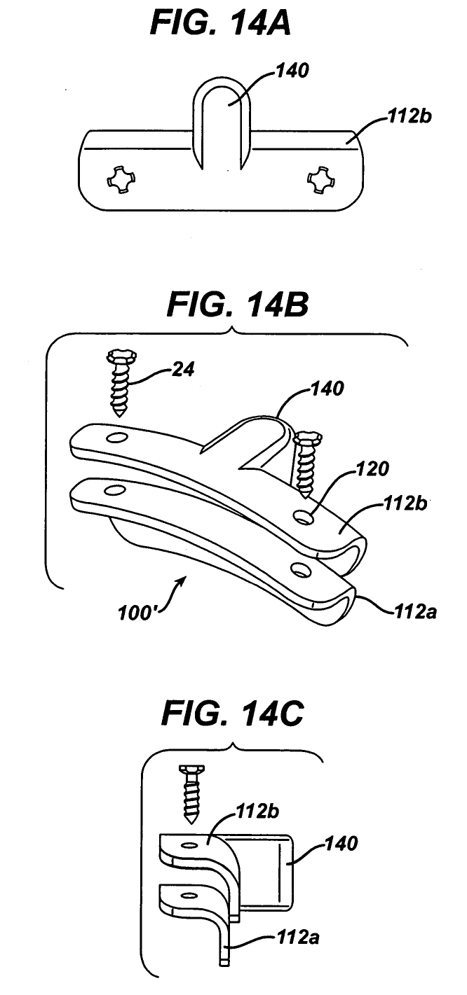

embodiment, the implant fixation device described herein can assist with retention of prosthetic discs or disc implants. For example, an extension portion 140 can be positioned on at least one of the plate bodies 112a, 112b such that when device 100′ is implanted, extension portion 140 extends into the space disc space between vertebral bodies. If a disc prosthesis tends to back out of the disc space it will encounter extension portion 140 and be prevented from further movement. FIGS. 14A through 14C illustrate one example of device 100′ with extension portion 140 extending from top plate body 112b. Extension portion 140 is sized and shaped such that when plate bodies 112a, 112b are implanted on a vertebral body, the extension portion will extend into the space between adjacent vertebral bodies. In one aspect, extension portion 140 is defined by a fin-like protrusion that extends from a portion of plate body 112b (e.g., it extends from second plate segment 134).

FIG. 15 illustrates device 100′ is implanted on an inferior corner of a vertebral body 70 such that extension portion 140 extend into the inferior disc space. While extension portion 140 is illustrated with respect to the two-plate-body embodiment of device 100, one skilled in the art will appreciate that extension portion 140 can similarly be applied to any of the embodiments described herein.

In another embodiment of the methods and apparatus described herein, a hinged device 200 is provided. Device 200 can include a plate body having two segments joined by a hinge, such that the two segments can pivot with respect to each other. A user can position an implant between the segments and then pivot at least one of the segments to pin the implant between the segments and thereby hold the implant in place. In one aspect, the plate body can include one, two, or more than two implant receiving openings. The implant can extend through the opening(s) and into an area between the two segments of the plate body. In an alternative embodiment, the plate body does not include any implant receiving openings. One skilled in the art will appreciate that the hinged plate can include the various features described above with respect to the devices 10, 100, 100′ including for example, apertures for receiving fixation devices, surface features for gripping the implant and/or native tissue, and/or features for mating the plate segments similar to those used to mate the two plate devices 100, 100′.

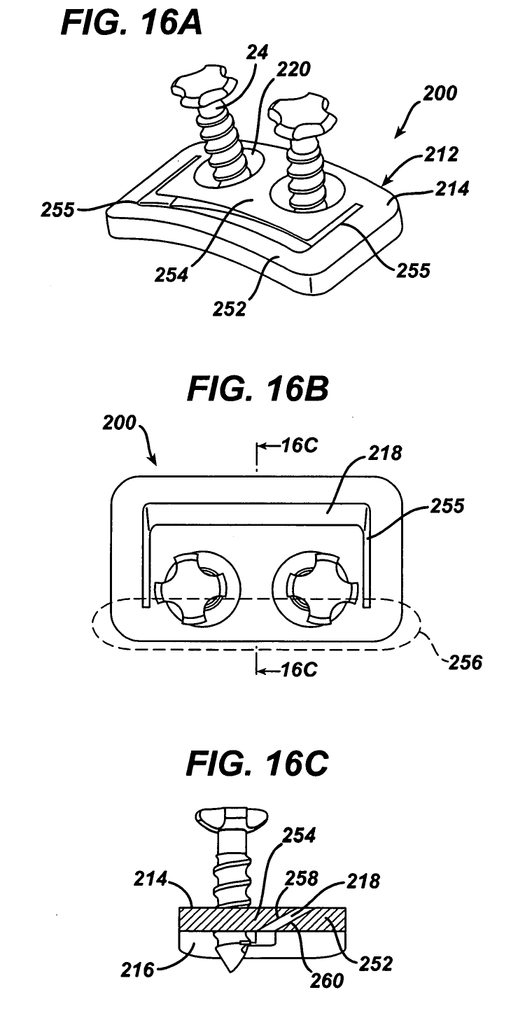

One embodiment of device 200 is illustrated in FIGS. 16A through 16C, including a single implant receiving opening 218 and apertures 220 for receiving fixation device. Plate body 212 has an upper surface 214 and a lower native tissue contacting surface 216 as well as a first plate body segment 252 and a second plate body segment 254. Segments 252, 254 are joined by hinge 256. Between segments 252, 254 is a slot 255, at least a portion of which defines implant receiving opening 218. Opening 218 allows an implant to be positioned such that it extends across a portion of lower plate surface 216, through implant receiving opening 218, and between plate segments 252, 254. Pivoting plate segment 252 toward plate segment 254 fixes the implant in position. Implant 222 can also be fixed by implanting plate body 218, with the implant located between lower surface 216 and native tissue.

As shown in FIG. 16B, slot 255 is defined by a gap between segments 252, 254. Segment 254 has a generally rectangular shape with one side connected to segment 252 via hinge 256. Slot 255 extends around three sides of segment 254 such that it has a generally “U” type shape. While an implant can be extended through any portion of slot 255, in one embodiment implant receiving opening 218 is defined by the longitudinally extending portion of slot 255.

As shown in the cross-sectional view of device 200 provided by FIG. 16C, opening 218 extends through plate body 212 at an angle with respect to top and bottom surface 214, 216. For example, opening 218 can extend through the plate body at a non-perpendicular angle with respect to the upper and/or lower surfaces of the device. This provides implant contact areas 258, 260 for gripping an implant. With an implant positioned between implant contact areas 258, 260, segments 252 and 254 can be brought together to fix the implant in position.

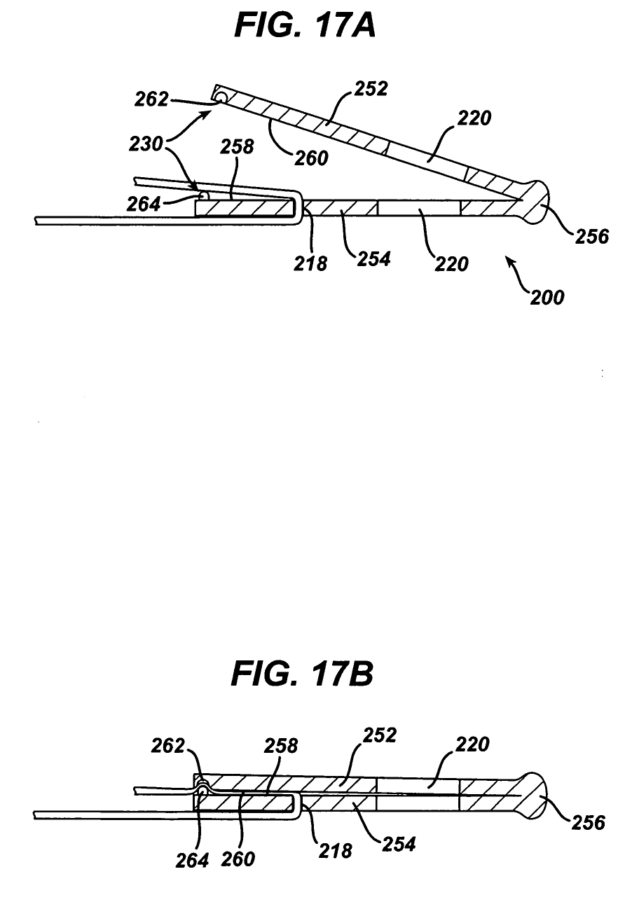

In an alternative embodiment, opening 218 can be positioned perpendicularly with respect to top and bottom plate surfaces 214, 216. FIGS. 17A and 17B illustrate plate body 212 with implant receiving opening 218 positioned in plate segment 254. An implant can extend through opening 218 and be fixed in position between contact areas 258, 260 of plate segments 252, 254.

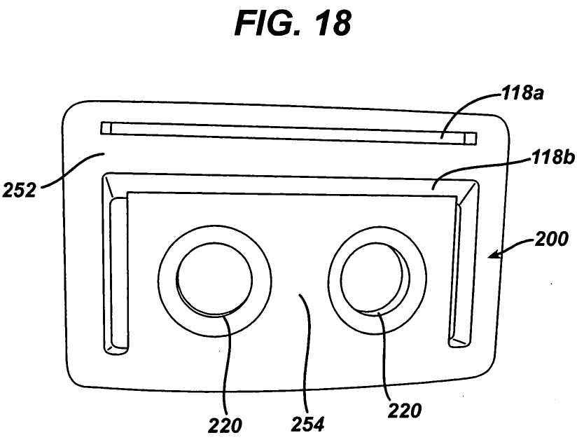

Implant receiving opening 218 of device 200 can be positioned in a variety of locations on plate body 212. For example, opening 218 can be defined by an elongate slot that runs parallel to hinge 256. More than one opening 218 can also be included in plate body 212. FIG. 18 illustrates plate body 212 with a first implant receiving opening 218a and a second implant receiving opening 218b. An implant can be threaded through first opening 218a between segments 252, 254 and then back through opening 218b, such that the implant wraps around a portion of body 212. One skilled in the art will appreciate that opening(s) 218 can have a variety of different sizes and shapes.

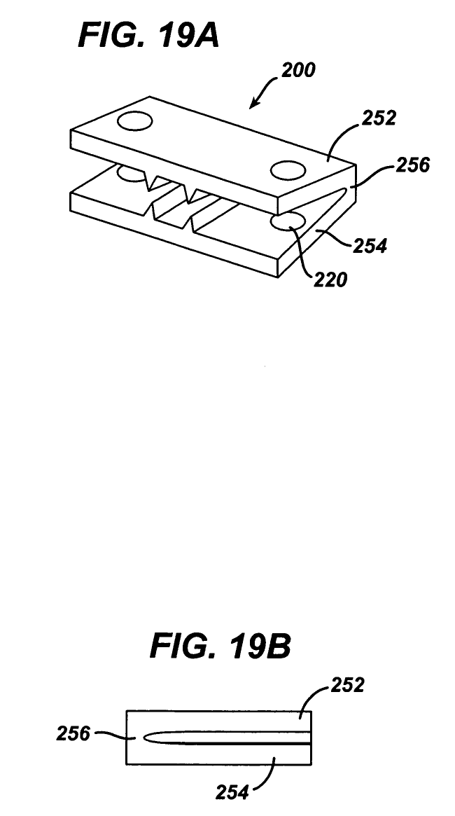

Alternatively, plate body 212 can be designed without an implant receiving opening. An implant can be laid between segments 252, 254 without extending through an implant receiving opening as shown in FIGS. 19A and 19B. The joinder of segments 252 and 254 will hold the implant therebetween.

Hinges 256 allow relative movement of segments 252, 254. A variety of hinges can be used with device 200, and in one embodiment, plate body 212 is a contiguous single body and hinge 256 is a living hinge. Living hinge 256 can be created by forming a thin area in plate body 212 that allows bending of plate body 212. One skilled in the art will appreciate that the creation of a living hinge will depend on a number of factors including the materials from which the plate body is created and the geometry of the plate body. In one embodiment plate body 212 is formed from materials that are flexible or pliable as described above.

Aperture(s) 220 in plate body 212 can be located such that when the fixation devices are implanted the fixation devices do not penetrate the implant. In addition, apertures 220 can be positioned such that when a fixation device is implanted through the aperture, it causes plate segments 252, 254 to pivot toward one another and fixes segments 252, 254 relative to one another. As shown in the cross-section view provided in FIG. 16C, apertures 220 extend through plate segment 252. When fixation devices are implanted through segment 252, segment 252 is pivoted toward segment 254, bringing the segments together and fixing an implant therebetween. Alternatively, apertures 220 can extend through both segments 252, 254. The cross-sectional view provided by FIGS. 17A and 17B shows aperture 220 extending through both segments 252, 254 and positioned such that when a fixation device is implanted it will fix segments 252, 254 relative to one another.

Segments 252, 254 can include surface features to assist with gripping implant 222. For example, segments 252, 254 as illustrated in FIGS. 17A and 17B include surface features 230 on implant contacting surfaces 258, 260. Implant contacting surface 260 includes a recess 262 that can receive a protrusion 264 on surface 258. When segment 252 is pivoted into contact with segment 254, at least a portion of implant 222 is forced into recess 262 by protrusion 264. One skilled in the art will appreciate that a variety of surface features can be used with device 200. In one embodiment, surface features are adapted to grip an implant without penetrating the implant. Alternatively, surface features can puncture the implant. The large surface area of implant contacting surfaces 258, 260 can provides support for implant 222 where it is pierced.

As mentioned above, the devices described herein can be used to fix a variety of implants. In one embodiment, the implant is a soft tissue implant formed from materials, such as, for example resorbable and non-resorbable polymers, allografts, autografts, xenografts, and combinations thereof. In one aspect, the implant is formed from graft materials, such as, for example tendenous, cartilaginous, ligamentous, protein or collagen based materials, extra- cellular matrices (ECMs), or other synthetic resorbable or non-resorbable graft materials. In yet another embodiment, the implant is formed from small intestine submucosa (SIS). The implant can also be reinforced/enhanced with a variety of materials to augment its natural properties and/or promote tissue growth. In one aspect, the implant can include a coating or laminate of resorbable polymers and/or be treated or coated with a variety of growth factors, anti-coagulants and/or lubricants. In addition, or alternatively, the implant can be oriented to minimize local soft tissue adhesion by positioning the implant such that the treated or luminal side (if ECM) is oriented toward local soft tissue.

Fixation of the implant can also be augmented with materials to enhance securement, apposition, integration, and/or to fill voids created by a device/implant recessed within a bone hole. Exemplary augmentation materials can include adhesives (e.g., fibrin, polymeric glues, etc.), bone void fillers (e.g., hydroxyapatite, tricalcium phosphate, DBM putty, bone cement, and combinations thereof, etc.), injectable bone substitutes (e.g., collagen, BMP, etc.), growth factor delivery systems (e.g., osteoconductive matrix formulations (Healos), recombinant human growth/differentiation factor-5 (MP52), etc.), and combinations thereof. Augmentation can also be in the form of sutures wrapped around the implant and/or device. An alternative method of augmentation can include a plug or a second bone anchor that can fill voids between, above, or below the implanted device.

One of ordinary skill in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A soft tissue graft implantation device, comprising:
a first plate including a body having an upper surface, a lower surface with an implant contacting area, and at least one aperture for receiving a fixation device; and
a second plate including a body having an upper surface with an implant contacting area,
a lower surface, and at least one aperture for receiving a fixation device,
wherein the first and second plates are adapted to fix an implant between the lower surface of the first plate and the upper surface of the second plate.

2. The device of claim 1, wherein at least one of the implant contacting areas of the first and second plates includes one or more surface features adapted to grip an implant without puncturing the implant.

3. The device of claim 1, wherein at least one of the first and second plates includes surface features for gripping an implant, the surface features being selected from the group consisting of barbs, tines, fins, ribs, ridges, a coating, and combinations thereof.

4. The device of claim 1, wherein the at least one aperture of the first and second plates is adapted to receive a fixation element selected from the group consisting of a pin, screw, staple, tine, wire, anchor, expansion bolt, rivet, and combinations thereof.

5. The device of claim 1, wherein the at least one aperture of the first and second plates is adapted to mate with a fixation element.

6. The device of claim 1, wherein the apertures are positioned on the plate such that implantation of a fixation element through the apertures will not penetrate an implant positioned between the first and second plates.

7. The device of claim 1, wherein at least one of the first and second plates includes a slot adapted to receive at least a portion of an implant.

8. The device of claim 1, wherein the slot in the at least one of the first and second plates extends between the upper surface and the lower surface of the plate at a non-perpendicular angle with respect to the upper surface and the lower surface of the plate.

9. The device of claim 1, wherein the first plate has a first mating feature and the second plate has a second mating feature adapted to mate with the first mating feature.

10. The device of claim 1, wherein at least a portion of the first plate is adapted to sit within a recess in the second plate.

11. The device of claim 1, wherein the first and second plates are flexible or malleable.

12. The device of claim 1, wherein the lower surface of the second plate has a contour that corresponds to the contour of a vertebral body.

13. The device of claim 12, wherein the first and second plates are adapted for implanting on a single vertebral body.

14. The device of claim 12, wherein at least one of the first and second plates includes a protrusion adapted to extend into the space between adjacent vertebral bodies.

15. The device of claim 1, wherein the first and second plates include multiple apertures, the multiple apertures positioned for implanting multiple fixation elements in a single vertebral body.

16. A method of implanting a soft tissue graft, comprising:
providing a first plate including a body having an upper surface and a lower implant contacting surface and a second plate including a body having an upper implant contacting surface and a lower tissue contacting surface;
positioning a first end of an implant between the first and second plates; and
fixing the first and second plates to a hard tissue surface, such that the implant is fixed between the first and second plates.

17. The method of claim 16, further comprising the step of implanting a second end of the implant with a second device.

18. The method of claim 16, further comprising the step of implanting the first and second plates on a first vertebral body.

19. The method of claim 18, further comprising the step of implanting a second set of first and second plates on a second vertebral body.

20. The method of claim 16, further comprising the step of positioning an implant between the lower surface of the second plate and a tissue surface.

21. The method of claim 16, further comprising the step of implanting fixation devices through the first and second plates without puncturing the implant.
